(19) United States
(12) Patent Application Publication
(10) Pub. No.: US 2008/0125747 A1
(43) Pub. Date: May 29, 2008

(54) PASSIVE THERMAL SPINE CATHETER

(75) Inventor: PAUL F. PROKOP, Woburn, MA (US)

Correspondence Address: FISH & RICHARDSON P.C.;Smith & Nephew, Inc., 1450 Brooks Road, Memphis, TN, 38116, US

(73) Assignee: Smith & Nephew, Inc.-TN, Memphis, TN (US)

(21) Appl. No.: 11/564,145

(22) Filed: Nov. 28, 2006

(51) Int. Cl. A61M 31/00; A61B 18/04

(52) U.S. Cl. 604/508; 606/28

(57) ABSTRACT

A method of treating a herniated intervertebral disc includes inserting a heat-transfer area of a tube containing a fluid into a spinal column and transferring heat between the tube and a tissue. Vapor flows from an evaporator zone of the tube to a condenser zone and liquid flows from the condenser zone to the evaporator zone. Insulation material can be removed from the tube to adjust the heat-transfer area of the tube. The tube is configured such that when a temperature of a heat portal area of the tube is changed fluid changes state and flows to the heat-transfer area. The tube also contains a wick structure to assist liquid flow by way of capillary action. In addition, the tube includes a flexible segment that conforms to the inner curved surface of the annulus fibrosus of the intervertebral disc.

### TECHNICAL FIELD

This description relates to a passive thermal spine catheter for treating spinal tissue.

### BACKGROUND

Spine catheters are used to treat damaged tissue within a spinal column. The spine includes vertebrae separated by intervertebral discs that join the vertebrae, providing flexibility and cushioning against impact. The intervertebral discs include an outer annular ring made of a fibrous material, the annulus fibrosus, that contains a soft inner material, the nucleus pulposus. As the annulus fibrosus weakens with age, or due to injury, the annulus fibrosus sometimes develops a tear, or fissure, and in some cases the nucleus pulposus becomes extruded through the fissure—a condition referred to as herniation. A herniated disc is also known as a ruptured, prolapsed, bulging, or slipped disc. The damaged disc sometimes applies pressure to nearby nerves in the spinal column, resulting in minor to disabling pain.

Catheters are also used to treat other tissues, such as connective tissues in joints, including the shoulder, knee and hip. For example, ligaments, tendons, muscles and cartilage form a capsule around the shoulder joint to maintain stability of the joint. Trauma or overuse can cause these soft tissues to stretch or tear, resulting in a condition known as shoulder instability, with accompanying pain and weakness.

### SUMMARY

According to one general aspect, a method includes inserting a heat-transfer area of an elongated tube into a spinal column and transferring heat between the tube and spinal tissue. Fluid, for example, water, within the tube flows from an evaporator zone of the tube to a condenser zone of the tube in a vapor state and fluid within the tube flows from the condenser zone to the evaporator zone in a liquid state.

Implementations of this aspect may include one or more of the following features. The method includes inserting the heat-transfer area of the tube into an intervertebral disc, and the tissue comprises a herniated area of the intervertebral disc. The method includes providing the heat-transfer area of the tube with a flexible segment that flexes to conform to an inner curved surface of an annulus fibrosus of the intervertebral disc.

The method includes adding heat to the tube at a heat portal area of the tube that is not inserted into the spinal column, and transferring heat from the tube to the tissue. Fluid vaporizes at the evaporator zone and condenses at the condenser zone, where the evaporator zone corresponds to the heat portal area and the condenser zone corresponds to the heat-transfer area. Heat is added at a rate calculated to maintain a predetermined temperature of an outer surface of the heat-transfer area of the tube. The rate is calculated, for example, to maintain the outer surface at about 90° C. The method includes receiving a temperature reading from a sensor located at the heat-transfer area of the tube and adjusting a rate at which heat is added in order to maintain the temperature reading within a predetermined tolerance around a temperature setting, for example, greater than about 75° C.

Heat is also removed from the tube at the heat portal area of the tube, in which case fluid vaporizes at the evaporator zone and condenses at the condenser zone, where the evaporator zone corresponds to the heat-transfer area and the condenser zone corresponds to the heat portal area. The method includes alternately adding heat to and removing heat from the tube at the heat portal area of the tube, heat being alternately transferred from the tube to the tissue and from the tissue to the tube.

According to another general aspect, a method includes removing a section of an insulation material from an elongated tube to expose a length of the tube, such that a variable-length heat-transfer area of the tube is adjusted. The tube contains fluid and is configured such that when a temperature of a heat portal area of the tube is changed, fluid changes state and flows to the heat-transfer area.

Implementations of this aspect may include one or more of the following features. Fluid changes from a first state to a second state at the heat portal area, and fluid changes from the second state to the first state at the heat-transfer area and flows to the heat portal area. The method includes inserting at least the heat-transfer area of the tube into a spinal column. The heat-transfer area includes the exposed length. Heat is transferred between the heat-transfer area of the tube and tissue, for example, a herniated area of the intervertebral disc.

According to another general aspect, a device includes an elongated tube configured for insertion into an intervertebral disc. The tube has a heat portal area and a heat-transfer area, such that when a temperature of the heat portal area is changed fluid contained within the tube changes state and flows to the heat-transfer area.

Implementations of this aspect may include one or more of the following features. The tube includes a flexible segment configured to conform to an inner curved surface of an annulus fibrosus of the intervertebral disc. The flexible segment includes a shape memory material that conforms to a predetermined shape when a second temperature of the heat-transfer area is increased above a predetermined level. In the device illustrated, embodiments include a removable insulation material around at least a portion of the tube, a wick structure inside the tube, and a heat source/sink coupled to the heat portal area of the tube.

Advantages may include one or more of rapidly heating or cooling spinal tissue, rapidly alternating between heating and cooling spinal tissue, heating or cooling a desired area of spinal tissue using a heating element having a selectable length, more uniform heating or cooling including increased control of the heating or cooling profile, heating or cooling spinal tissue without introducing an electrical current or a potentially harmful fluid into a patient's body, and reducing collateral damage to other bodily tissues.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is an illustration of a passive thermal medical device shown inserted into an intervertebral disc.

FIG. 2 is a cutaway view of the device of FIG. 1 in operation.

FIG. 3 is an illustration of the device of FIG. 1 having an insulation material partially removed.

FIG. 4 is a cutaway view of a bellows-type flexible segment of the device of FIG. 1.

FIG. 5 is a cutaway view of a slotted flexible segment of the device of FIG. 1.

FIG. 6 is a partially cutaway view of a coil-type flexible segment of the device of FIG. 1.

FIG. 7 is a graph comparing a surface temperature gradient of the device of FIG. 1 to that of another thermal device.

FIG. 8 is a graph comparing a tissue temperature surrounding the device of FIG. 1 and that surrounding the other thermal device.

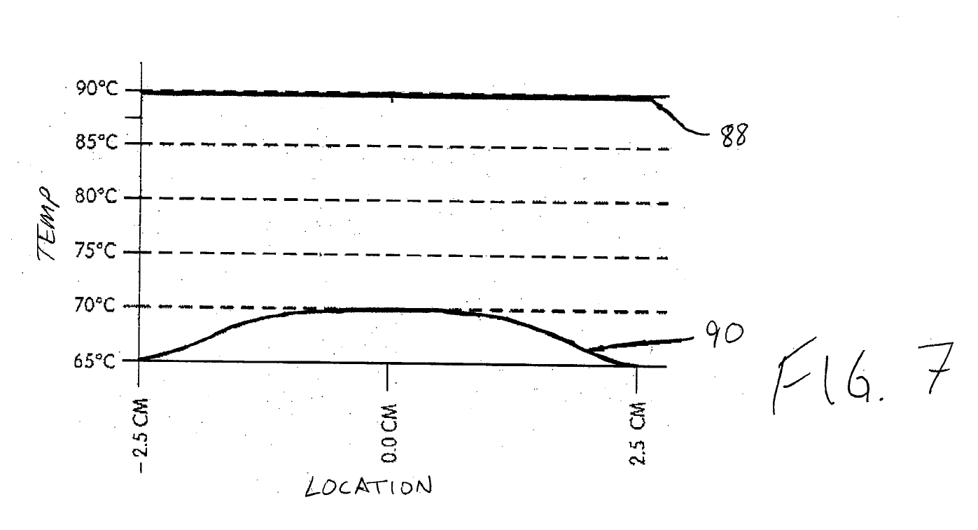

FIG. 6 is a block diagram of a method of treating a herniated intervertebral disc.

### DETAILED DESCRIPTION

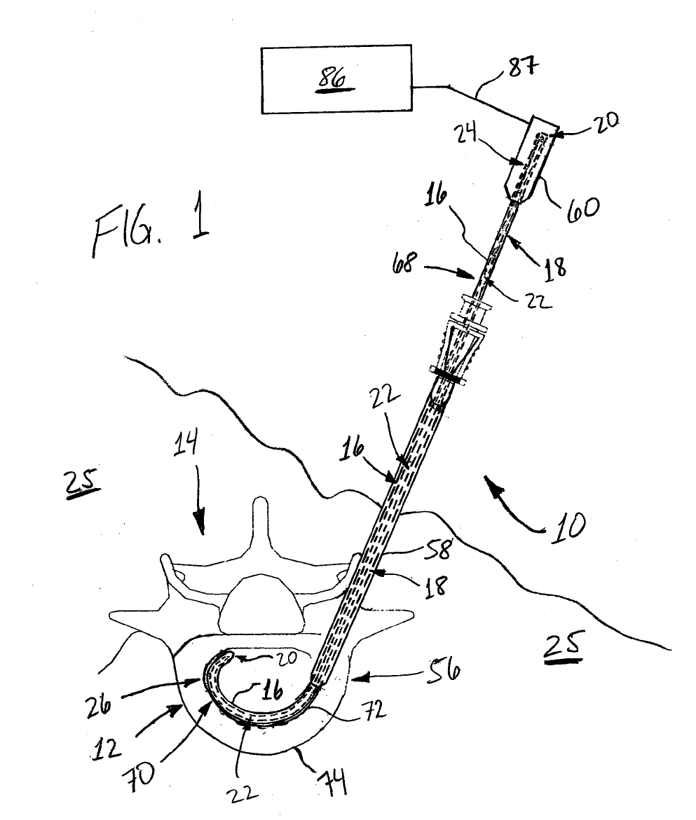

As illustrated in FIG. 1, a medical device 10 for treating tissue 12 in a spinal column 14 includes an elongated tube 16 defining a lumen 18 containing a fluid. The tube 16 is sealed at both ends 20 and evacuated so that in a normal operating temperature range of about negative fifty to about positive one hundred degrees Celsius (° C.) some of the fluid takes the form of a vapor and some of the fluid takes the form of a liquid. By adding heat to a localized area of the tube 16 and removing heat from another area of the tube 16, a vaporization-condensation cycle is created in which some of the fluid vaporizes in the heated area of the lumen 18 and flows toward the cooled area of the lumen 18, where the fluid condenses and then flows back toward the heated area. Inside the lumen 18 is a wick structure 22 that facilitates capillary motion of the liquid along the lumen 18 from the cooled area to the heated area.

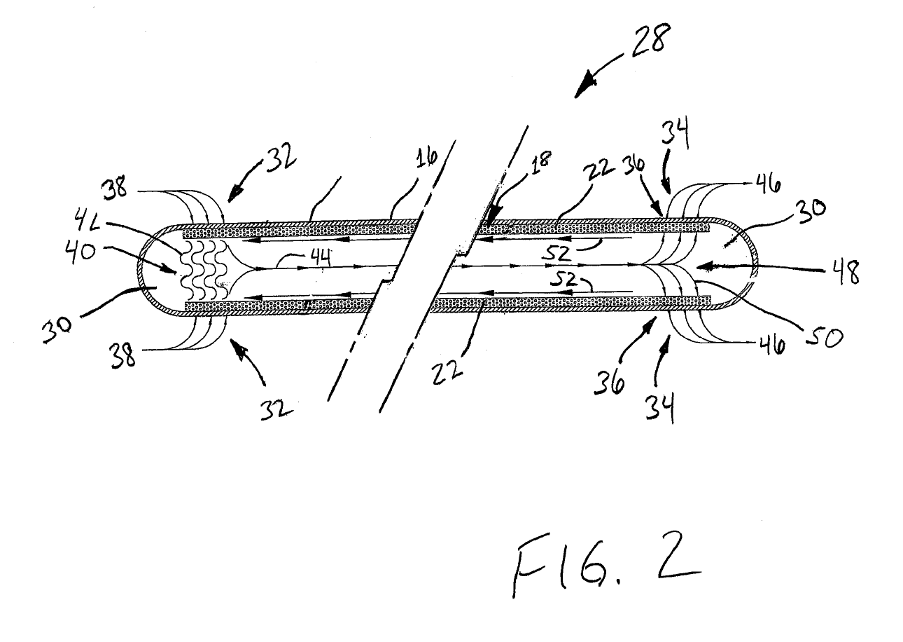

The tube 16 forms a heat pipe 28 (FIG. 2), with heat being transported by the cycling fluid. Heat is added or removed at a heat portal area 24 of the tube 16 outside a patient's body 25, and transferred out (when added at area 24) or transferred in (when removed at area 24) at a heat-transfer area 26 of the tube 16 positioned adjacent tissue 12. This results in heating of tissue 12 when heat is transferred out and cooling of tissue 12 when heat is transferred in, or alternately heating and cooling of tissue 12.

As shown in FIG. 2, when the heat pipe 28 is used for heating tissue 12, working fluid 30, or charging fluid, inside the heat pipe 28 vaporizes as it absorbs heat in an evaporator zone 32 of the heat pipe 28 at the heat portal area 24 of the tube 16 and condenses as it releases heat in a condenser zone 34 of the heat pipe 28 at the heat-transfer area 26 of the tube 16 in a continuous cycle. When the heat pipe 28 is used for cooling tissue 12, the cycle reverses, and working fluid 30 vaporizes as it absorbs heat in the evaporator zone 32 at the heat-transfer area 26 and condenses as it releases heat in the condenser zone 34 at the heat portal area 24. In both the heating and cooling cycles, the quantity of heat absorbed and released is equal to the latent heat, or heat of transition, of the mass of fluid 30 that is vaporized and condensed, plus the heat required to effect any accompanying temperature change of the fluid 30. By way of this mechanism, the heat pipe 28 transports heat from the evaporator zone 32 to the condenser zone 34 of the heat pipe 28, while maintaining a minimal temperature differential, or temperature gradient, between the two zones.

The tube 16 is evacuated and an appropriate quantity of fluid 30 is used to ensure that two phases of the fluid 30—liquid and gas—coexist in equilibrium throughout the normal operating temperature range of the heat pipe 28. At a lower end of the temperature range, a relatively high proportion of the fluid 30 exists in a liquid state and a relatively low proportion of the fluid 30 exists in a vapor state. Conversely, at the upper end of the temperature range, a relatively low proportion of the fluid 30 remains in the liquid state and a relatively high proportion of the fluid 30 is in the vapor state. As a result, the internal pressure in the lumen 18 at any given point in time is equal to the saturation pressure of the fluid 30 at the current temperature.

As a result, when heat is added (arrows 38) to the tube 16 at the evaporator zone 32, some of the fluid 30 in the evaporator zone 32 vaporizes 40, transitioning from a liquid state, or phase, to a vapor state. The quantity of heat absorbed by the fluid 30 is equal to the heat of vaporization of the mass of fluid 30 that is vaporized 40, plus any accompanying temperature change of the fluid 30. In order to maintain pressure equilibrium throughout the lumen 18, the resulting vapor 42 flows away 44 from the evaporator zone 32. In this manner, a rapid mass transfer of fluid 30 occurs, with a corresponding energy displacement.

Simultaneously, heat is removed (arrows 46) from the tube 16 at the condenser zone 34, and some of the fluid 30 in the condenser zone 34 condenses 48, transitioning from the vapor state to the liquid state. The quantity of heat removed 46 from the fluid 30 is equal to the heat of condensation of the mass of fluid 30 that is condensed 48, plus any accompanying temperature change of the fluid 30. The condensate, or liquid 50, is absorbed by the wick structure 22 and transported by capillary action 52 through the wick structure 22 toward the evaporator zone 32, where liquid 50 from the wick structure 22 is vaporized 40, completing the cycle. In this manner, as long as heat is added 38 and removed 46, a continuous vaporization-condensation cycle is sustained in the heat pipe 28.

The temperature of the heat pipe 28 is adjusted by varying the rates at which heat is added 38 and removed 46. If the rate at which heat is removed 46 at the condenser zone 34 differs from the rate at which heat is added 38 at the evaporator zone 32, the temperature of the working fluid 30 in the heat pipe 28 changes as thermal energy either accumulates (in the case that heat is added 38 at a greater rate than heat is removed 46) or diminishes (in the case that heat is added 38 at a lesser rate than heat is removed 46) in the working fluid 30. However, if the rates are equal, the temperature of the working fluid 30 remains constant while heat is added 44 and removed 46 in an isothermal process.

The heat pipe 28 is well-suited for use in invasive medical treatments, since it does not have any moving parts or electrical conductors. Heat pipes have been proven reliable for over 100,000 hours of continuous use. In addition, the medical device 10 has fewer parts and is less costly to manufacture than a typical spine catheter that contains a heating element. Furthermore, a non-toxic working fluid 30, such as distilled water, can be selected. Moreover, since the tube 16 is under vacuum, if the heat pipe seal is broken, surrounding air or fluid typically leaks into the heat pipe 28, rather than the working fluid 30 leaking out.

Referring again to FIG. 1, the tube 16 extends from the heat portal area 24 to the heat-transfer area 26, each of which includes an exposed length of metal, such as copper. The tube 16 is made of a metal, such as copper, and has a total length of, for example, from about 30 to about 50 centimeters. Furthermore, in order to guide the tube 16 into a patient's body 54, into the spinal column 14 and eventually into an intervertebral disc 56, the medical device 10 includes an introduction needle 58. The wick structure 22, for example, a copper mesh, extends from the heat portal area 24 to the heat-transfer area 26 to facilitate capillary motion 52 (see FIG. 2) of the liquid 50 between the heat portal area 24 and the heat-transfer area 26.

The medical device 10 includes a heat source/sink 60 that communicates with the heat portal area 24 of the tube 16 to conduct a measured quantity of heat to or from the heat portal area 24 of the tube 16 at a location outside of a patient's body 54. In one implementation of the medical device 10, the heat source/sink 60 is a radio frequency (RF) heat source, such as the Smith & Nephew ELECTROTHERMAL™ 20S Spine System, which interfaces with the heat portal area 24. In this implementation, the heat source/sink 60 generates heat, which is conducted to the heat portal area 24 of the tube 16.

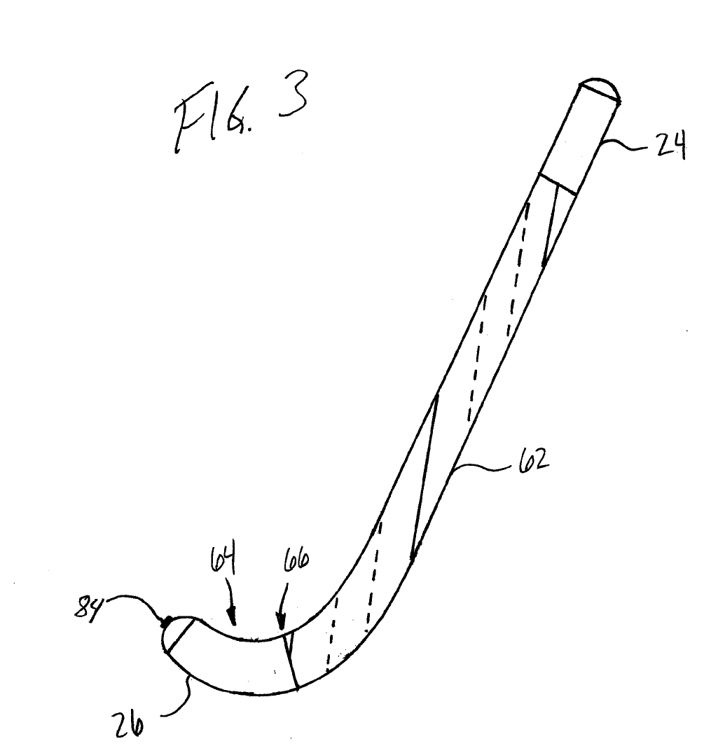

Referring to FIG. 3, with the exception of the heat portal area 24 and the heat-transfer area 26, the tube 16 is coated with an insulation material 62 to thermally insulate the tube 16, thus minimizing conduction or convection of heat to or from bodily tissues other than the targeted spinal tissue 12. In addition, the insulation material 62 minimizes heat loss from the portion of the tube 16 outside the patient's body 54 during the heating cycle (and heat gain during the cooling cycle). The heat-transfer area 26 has an exposed length 64 that can be varied by removing insulation material 62. The removable insulation material 62 thus forms a variable-length heat-transfer area 26 that can be varied, for example, from a length of about one to about ten centimeters. In FIG. 3, the insulation material 62 has been removed beyond an edge 66 to reveal a desired exposed length 64 that interfaces with spinal tissue 12.

Referring again to FIG. 1, the tube 16 has a rigid or semi-rigid body 68 along most of its length, but in the general vicinity of the heat-transfer area 26, the tube 16 includes a flexible segment 70 that conforms to a curved inner wall 72 of an annulus fibrosis 74 of the intervertebral disc 56 in the spinal column 14. For example, the flexible segment 70 has a is length of about one to about ten centimeters, and conforms to a radius of curvature of about one to about five centimeters. As shown in FIG. 4, the flexible segment 70 is formed from a length of, for example, copper tubing having a bellows shape 76. The flexible segment 70 can also be coated with an outer layer 78 of polytetrafluoroethylene (PTFE), or Teflon™, to seal and insulate the tube 16. However, the heat-transfer area 26 remains exposed to facilitate conduction or convection of heat to the surrounding tissue 12 (see FIG. 1).

Referring again to FIG. 3, a temperature sensor 84, such as a thermocouple, is installed, for example, on the outer surface of the heat-transfer area 26 of the tube 16. A processor 86, shown in FIG. 1, receives a temperature reading, or signal, from the temperature sensor 84 and performs an algorithm to determine a heat rate to maintain the temperature of the heat-transfer area 26 at or within an acceptable tolerance from a preselected temperature setting, or setpoint. The processor 86 is linked to the heat source/sink 60, for example, by way of an electrical wiring 87, in order to control the heat source/sink 60.

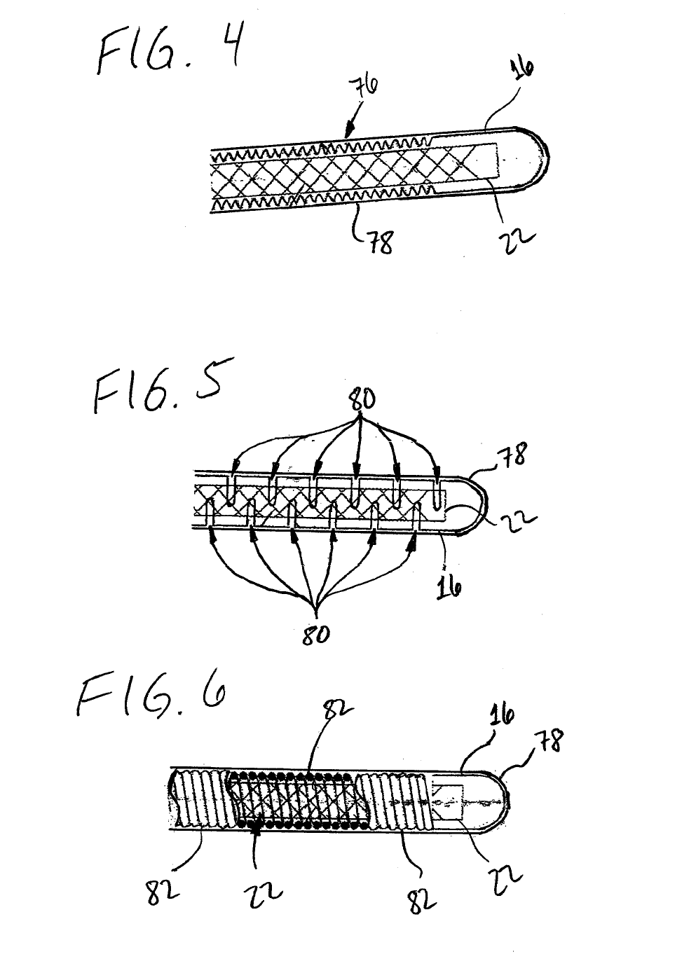

Other embodiments are within the scope of the following claims. For example, referring to FIG. 5, in another implementation of the medical device 10, the flexible segment 70 is formed by cutting a series of slots 80, or perforations, along a length of the tube 16, for example, perpendicular to the axis of the tube 16. The slots 80 are cut, for example, using a laser, EDM, or mechanical machining methods. Alternatively, the slots 80 can be cut in other shapes or orientations, such as a curved shape or at an oblique orientation with regard to the axis of the tube 16. Moreover, as illustrated in FIG. 6, in yet another implementation of the medical device 10, the flexible segment 70 is formed from a coil spring 82 made of stainless steel. In this implementation, the wick structure 22 passes inside the spring 82, and the outer layer 78 is sealed around the spring 82 to contain the fluid 30 and insulate the tube 16. The heat-transfer area 26 remains exposed to facilitate conduction and convection of heat to the surrounding tissue 12.

Alternatively, the tube 16, including the rigid or semi-rigid body 68 and the flexible segment 70, can be made of a different metal, such as a stainless steel or a nickel-titanium (Ni—Ti) alloy, or a plastic material, PTFE or polyimide. The heat portal area 24 and/or the heat-transfer area 26 can made from a different material than the body 68, including, for example, a metal having greater thermal conductivity, such as copper or aluminum. In addition, the rigid or semi-rigid body 68 and the flexible segment 70 can be made of two different materials. Furthermore, the outer layer 78 can also be made of a plastic, polyimide or Kapton.

The working fluid 30 can be selected to provide advantageous properties, such as high latent heat to transport a relatively large quantity of energy with respect to mass flow, or vaporization temperature and vapor pressure characteristics that result in an acceptable liquid to vapor ratio throughout the intended operating temperature range of the heat pipe 28. For example, the working fluid 30 can be water, alcohol, ammonia, helium, mercury, a refrigerant, or any other fluid capable of vaporization to complete the thermal cycle.

The wick structure 22 can be made from a different metal, such as a stainless steel or a Ni—Ti alloy, or from a sintered metal powder. In general, the wick structure 22 can be made from any material capable of soaking up the fluid 30. The wick structure 22 can be made from a wire mesh, a screen or a series of grooves along the inner surface of the lumen 18 parallel to the axis of the tube 16.

The heat source/sink 60 can include a resistive heating element, a heat exchanger, a heat pump, a flame, or any other suitable heating device. Alternatively, the heat source/sink 60 can include a thermoelectric (Peltier) cooler, a chiller unit, a cold gel pack, or any other suitable cooling device.

The temperature sensor 84 can be located at a generally central location of the heat-transfer area 26. In implementations that do not include a temperature sensor 84, the amount and rate of heat added 38 or removed 46 at the heat portal area 24 can be determined by an algorithm, for example, calculated to maintain the tissue 12 at a therapeutic temperature for a treatment time period.

In addition, the medical device 10 can be adapted for use in thermal capsulorrhapy, the treatment of connective joint tissues, for example, in the shoulder, knee or hip. The shape, dimensions and materials of the tube 16 can be modified to facilitate insertion of the device 10 into a joint to treat the ligaments, tendons, muscles and cartilage that connect the bones of the joint. For example, the rigid or semi-rigid body 68 can extend the entire length of the tube 16 without including the flexible segment 70.

Referring generally to FIGS. 1 through 3, in use, a physician or medical technician removes a length of insulation material 62 from a portion of the heat pipe 28 corresponding to the tissue 12 to be treated in a particular patient and inserts the heat-transfer area of the tube 16 into an intervertebral disc 56 in the spinal column 14 using an introduction needle 58. As the heat pipe 28 is inserted, the flexible segment 70 of the tube 16 curves to conform to the inner wall 72 of the annulus fibrosis 74. The heat source/sink 60 adds heat 38 to the heat portal area 24 of the tube 16, which causes working fluid 30 in the heat pipe 28 to vaporize 40 and flow away 44 from the heat portal area 24 toward the lower-temperature, lower-pressure heat-transfer area 26 of the heat pipe 28. As heat is released, the fluid 30 condenses 48 and is absorbed by the wick structure 22 that transports the liquid 50 back to the heat portal area 24. That is, the heat portal area 24 acts as the evaporator zone 32 and the heat-transfer area 26 acts as the condenser zone 34.

Initially, as heat is added 38 at the heat portal area 24 the temperature of the tube 16 increases. Since the heat-transfer area 26 is in close proximity to or in contact with the inner wall 72 of the annulus fibrosis 74, to the extent that the outer surface of the heat-transfer area 26 of the tube 16 differs from that of the surrounding tissue 12, heat transfers from the exposed outer surface of the heat pipe 28 into the tissue 12 of the annulus fibrosus 74. When the temperature difference between the outer surface of the heat-transfer area 26 and the tissue 12 is sufficient to transfer heat at the same rate that heat is added 38 at the heat portal area 24, the device 10 reaches a steady-state isothermal condition.

Subsequently, the heat source/sink 60 removes heat 46 from the heat portal area 24 and the heat pipe cycle reverses, that is, the heat portal area 24 acts as the condenser zone 34 and the heat-transfer area 26 acts as the evaporator zone 32. Thus, fluid 30 releases heat and condenses 48 at the heat portal area 24. The wick structure 22 absorbs the condensate and transports the liquid 50 to the higher-temperature, higher-pressure heat-transfer area 26, where heat is transferred from the tissue 12 through the wall 36 of the tube 16 to the fluid 30, causing the tissue to be cooled. The fluid 30 in the heat-transfer area 26 absorbs the heat, vaporizes 40 and flows 44 back to the heat portal area 24.

In an implementation that includes a temperature sensor 84, the processor 86 receives the temperature reading and determines the appropriate heat rate to maintain the temperature of the heat-transfer area 26 at or within an acceptable tolerance from the temperature setpoint. The processor 86 controls the heat source/sink 60 to adjust the rate of heat added to or removed from the heat pipe as necessary to maintain the temperature setting.

Thus, the tissue 12 can be either heated or cooled using the device 10. Furthermore, the heat pipe cycle can be quickly reversed, resulting in an almost instantaneous transition from heating to cooling and vice versa. Thus, the medical device 10 can rapidly pulse between heating and cooling modes.

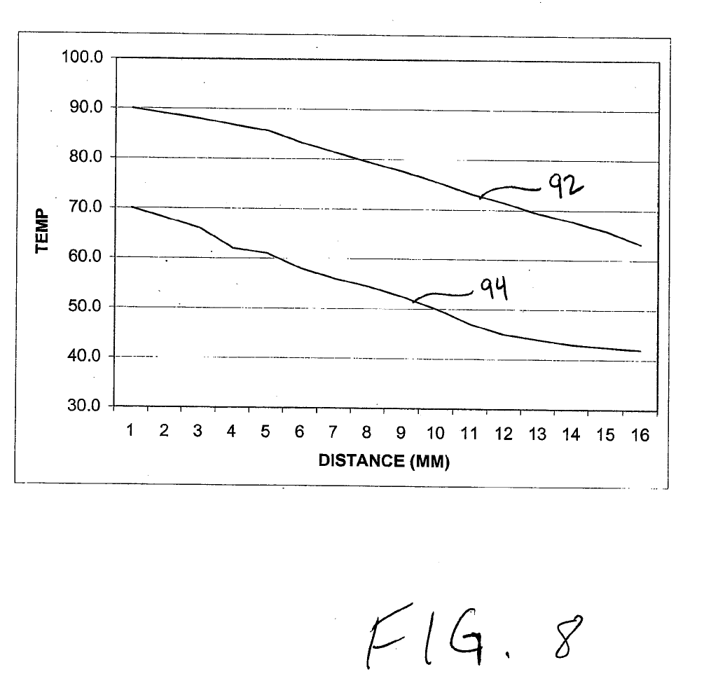

Use of the medical device 10 results in improved thermal characteristics with respect to a spine catheter that contains a heating element, such as a resistive heat coil, in the portion of the catheter that is inserted into the spinal column 14. For example, the graph of FIG. 7 contrasts a modeled surface temperature 88 along the length of a five-centimeter heat-transfer area 26 of the device 10 with a typical surface temperature 90 of the spine catheter that contains a heating element, assuming a temperature setpoint of about 90° C. The device 10 not only maintains the surface temperature 88 closer to the setpoint, but also maintains a more constant surface temperature 88 throughout the heat-transfer area 26. Similarly, the graph of FIG. 8 demonstrates a corresponding modeled thermal spreading 92 through the annulus fibrosus 74 using the device 10 versus a typical thermal spreading 94 attained using the spine catheter that contains the heating element. As a result of improved thermal spreading 92, the device 10 exposes more tissue 12 to a therapeutic temperature.

It will be understood that various modifications may be made. For example, useful results still could be achieved if steps of the disclosed techniques were performed in a different order and/or if components in the disclosed systems were combined in a different manner and/or replaced or supplemented by other components. Accordingly, other implementations are within the scope of the following claims.

What is claimed is:

1. A method, comprising:
inserting at least a heat-transfer area of an elongated tube into a spinal column; and
transferring heat between the tube and spinal tissue, wherein fluid within the tube flows from an evaporator zone of the tube to a condenser zone of the tube in a vapor state and fluid within the tube flows from the condenser zone to the evaporator zone in a liquid state.

2. The method of claim 1, further comprising providing the heat-transfer area of the tube with a flexible segment.

3. The method of claim 1, further comprising inserting the heat-transfer area of the tube into an intervertebral disc, wherein the tissue comprises a herniated area of the intervertebral disc.

4. The method of claim 3, further comprising providing the heat-transfer area of the tube with a flexible segment that flexes to conform to an inner curved surface of an annulus fibrosus of the intervertebral disc.

5. The method of claim 1, further comprising:
adding heat to the tube at a heat portal area of the tube that is not inserted into the spinal column; and
transferring heat from the tube to the tissue.

6. The method of claim 5, wherein fluid vaporizes at the evaporator zone and condenses at the condenser zone, the evaporator zone corresponding to the heat portal area and the condenser zone corresponding to the heat-transfer area.

7. The method of claim 5, wherein heat is added at a rate calculated to maintain a predetermined temperature of an outer surface of the heat-transfer area of the tube.

8. The method of claim 7, wherein the rate is calculated to maintain the outer surface at about ninety degrees Celsius.

9. The method of claim 5, further comprising:
receiving a temperature reading from a sensor located at the heat-transfer area of the tube;
adjusting a rate at which heat is added in order to maintain the temperature reading within a predetermined tolerance around a temperature setting.

10. The method of claim 9, wherein the temperature setting is greater than about 75 degrees Celsius.

11. The method of claim 1, further comprising removing heat from the tube at a heat portal area of the tube that is not inserted into the spinal column, wherein heat is transferred from the tissue to the tube.

12. The method of claim 11, wherein fluid vaporizes at the evaporator zone and condenses at the condenser zone, the evaporator zone corresponding to the heat-transfer area and the condenser zone corresponding to the heat portal area.

13. The method of claim 1, further comprising alternately adding heat to and removing heat from the tube at a heat portal area of the tube that is not inserted into the spinal column, wherein heat alternately is transferred from the tube to the tissue and from the tissue to the tube.

14. The method of claim 13, wherein fluid vaporizes at the evaporator zone and condenses at the condenser zone, the evaporator zone corresponding to the heat portal area and the condenser zone corresponding to the heat-transfer area when heat is added, and the evaporator zone corresponding to the heat-transfer area and the condenser zone corresponding to the heat portal area when heat is removed.

15. The method of claim 1, wherein the tube contains water.

16. A method, comprising:
removing a section of an insulation material from an elongated tube to expose a length of the tube such that a variable-length heat-transfer area of the tube is adjusted, wherein the tube contains fluid and is configured such that when a temperature of a heat portal area of the tube is changed fluid changes state and flows to the heat-transfer area.

17. The method of claim 16, wherein fluid changes from a first state to a second state at the heat portal area, and fluid changes from the second state to the first state at the heat-transfer area and flows to the heat portal area.

18. The method of claim 16, further comprising inserting at least the heat-transfer area of the tube into a spinal column, wherein the heat-transfer area includes the exposed length.

19. The method of claim 16, further comprising transferring heat between the heat-transfer area of the tube and a tissue.

20. The method of claim 18, further comprising inserting the heat-transfer area of the tube into an intervertebral disc, wherein the tissue comprises a herniated area of the intervertebral disc.

21. A device, comprising:
an elongated tube having a heat portal area and a heat-transfer area, the tube being configured for insertion into an intervertebral disc;
fluid contained within the tube, such that when a temperature of the heat portal area is changed fluid changes state and flows to the heat-transfer area.

22. The device of claim 21, wherein the tube comprises a flexible segment configured to conform to an inner curved surface of an annulus fibrosus of the intervertebral disc.

23. The device of claim 22, wherein the flexible segment comprises a shape memory material that conforms to a predetermined shape when a temperature of the heat-transfer area is increased above a predetermined level.

24. The device of claim 21, further comprising a removable insulation material around at least a portion of the tube.

25. The device of claim 21, further comprising a wick structure inside the tube.

26. The device of claim 21, further comprising a heat source or a heat sink coupled to the heat portal area of the tube.

27. A device, comprising:
an elongated tube having an evaporator zone and a condenser zone, the tube being configured for insertion into a spinal column to transfer heat between the tube and spinal tissue; and
fluid contained within the tube, wherein fluid flows from the evaporator zone of the tube to the condenser zone of the tube in a vapor state and fluid within the tube flows from the condenser zone to the evaporator zone in a liquid state.
